(19) United States
(12) Patent Application Publication
(10) Pub. No.: US 2017/0079321 A1
(43) Pub. Date: Mar. 23, 2017

(54) ELECTRICALLY-ACTUATED VAPORIZATION DEVICE FOR INGESTIBLE COMPOUNDS

(71) Applicant: Tyler Golz, Boulder, CO (US)

(72) Inventor: Tyler Golz, Boulder, CO (US)

(21) Appl. No.: 14/857,239

(22) Filed: Sep. 17, 2015

(51) Int. Cl. A24F 47/00; A24F 1/32; A24F 5/00; A24F 1/30; A61M 11/04; H05B 3/00

(52) U.S. Cl. CPC A24F 47/008; A61M 11/042; H05B 3/0014; A24F 5/00; A24F 1/30; A24F 1/32

(57) ABSTRACT

Embodiments directed to a disposable vaporizing assembly for vaporizing an ingestible compound and systems and methods related thereto. In one aspect, an embodiment including an electrically non-conductive bowl that receives an ingestible compound through an opening of the bowl for disposition relative to a base surface of the bowl. The base surface may be tapered to distribute the ingestible compound towards an interior surface of a wall of the electrically non-conductive bowl. The embodiments may further include an electrically conductive apparatus disposed over an exterior surface of the wall of the electrically non-conductive bowl and an actuation assembly interconnected to the electrically conductive apparatus. In this regard, the actuation assembly may electrically energize the electrically conductive apparatus to heat the electrically non-conductive bowl for vaporizing the ingestible compound.

### BACKGROUND

1. Field of the Invention

The present invention generally relates to vaporizing an ingestible compound, for example, to facilitate ingestion of a compound for various therapies of interest.

2. Relevant Background

Various therapies of interest (e.g., aromatherapy, respiratory therapy, smoking, and/or other medicinal or recreational therapies, etc.) may involve the ingestion (e.g., inhalation) of an ingestible compound in a gaseous state. In this regard, a solid or liquid-state ingestible compound for use with a given therapy of interest may undergo a phase change into a gas (e.g., via the application of heat to the ingestible compound) in order to produce vaporized (i.e., atomized) molecules of the ingestible compound for ingestion by a user. The efficacy of the various therapies of interest may depend at least in part on the purity of the gaseous mixture ingested by the user (e.g., the extent to which an inhaled volume of gas contains the vaporized molecules of the ingestible compound versus other extraneous or diluting elements, etc.). As such, it may be desirable to cause the ingestible compound to undergo a phase change into a gas free from extraneous (e.g., combustion-produced) chemical reactions (e.g., free from chemical reactions other than the oxidation and/or decomposition of hydrocarbon substances into CO₂ and H₂O). Further, it may be desirable to cause the ingestible compound to undergo a phase change into a gas in a manner involving minimal intervention from a user to facilitate a consistent, repeatable vaporization of a given ingestible compound, which may help support the efficacy of a given therapy of interest.

### SUMMARY

In view of the foregoing, a primary objective of the present disclosure is to provide a vaporizing assembly and corresponding system for use therein, thereby facilitating the ingestion of vaporized molecules of an ingestible compound by vaporizing an ingestible compound in a clean and efficient manner.

In one aspect, the present disclosure includes a disposable for vaporizing an ingestible compound. The disposable includes an electrically non-conductive bowl that includes a wall having opposite first and second ends and opposite interior and exterior surfaces, an opening adjacent the first end of the wall, a base portion adjacent the second end of the wall, a cavity defined by the base portion and the interior surface of the wall, and an axis extending through the opening, the cavity and the base portion. In this regard, the base portion includes a base surface that tapers from the second end of the wall towards the first end of the wall to distribute an ingestible compound received through the opening onto the base surface towards the interior surface of the wall. Notably, the base surface may be closed across a maximum cross-dimension of the cavity. The disposable further includes an electrically conductive apparatus disposed over the exterior surface of the wall of the electrically non-conductive bowl. The disposable further includes an actuation assembly electrically interconnected to the electrically conductive apparatus that electrically energizes the electrically conductive apparatus to heat the electrically non-conductive bowl for vaporizing the ingestible compound.

A number of feature refinements and additional features are applicable in the first aspect and contemplated in light of the present disclosure. These feature refinements and additional features may be used individually or in any combination. As such, each of the following features that will be discussed may be, but are not required to be, used with any other feature combination of the first aspect.

For example, in an embodiment, the electrically conductive apparatus may be wrapped over the exterior surface of the wall about the axis. In some instances, the electrically conductive apparatus may be spiral wound over the exterior surface of the wall about the axis. In either event, the electrically conductive apparatus may be a flat wire.

In another embodiment, the actuation assembly may include a first electrically conductive path electrically connected to a first end of the electrically conductive apparatus and a second electrically conductive path electrically connected to a second end of the electrically conductive apparatus. In this regard, the actuation assembly may further include an electrically conductive base member having a first cavity that receives a portion of the electrically non-conductive bowl such that the electrically conductive base member defines a portion of the first conductive path.

Further, the actuation assembly may also include at least one electrically non-conductive fastener receivable through an aperture in the portion of the bowl and an aligned bore in the base member for securing the electrically non-conductive bowl to the base member such that the electrically conductive apparatus may be electrically interconnected with the first conductive path of the actuation assembly. In this regard, the bore of the base member may be defined by an electrically conductive inner surface that defines a portion of the first conductive path. In turn, the electrically conductive inner surface may be defined by an electrically conductive insert that may be received within the bore.

In another embodiment, the actuation assembly may further include an electrically conductive spacer member disposed between the portion of the bowl and the base member such that the electrically conductive spacer member defines a portion of the first conductive path. In this regard, the electrically conductive spacer member may be in electrical contact with the first end of the electrically conductive apparatus. In some instances, the actuation assembly may further include a first conductive post that may be received in a second cavity of the base member such that the first conductive post defines a portion of the first conductive path.

In yet another embodiment, the axis of the bowl may extend through the first and second cavities of the base member. In this regard, a longitudinal axis of the at least one fastener may be perpendicular to the axis of the bowl. In some instances, the actuation assembly may further include an insulating member that electrically isolates the first and second conductive paths. As such, the insulating member may be an insulating sleeve disposed about the electrically conductive apparatus. In this regard, a gap may be defined between the insulating member and the electrically non-conductive bowl. For example, the gap may be defined about a substantial entirety of a circumference of the electrically non-conductive bowl.

According to another embodiment, the actuation assembly may further include a conductive member secured to an outside surface of the insulating member opposite the electrically conductive apparatus such that the conductive member defines a portion of the second conductive path. In turn, the actuation assembly may further include an electrically conductive lead electrically connected between the conductive member and the second end of the electrically conductive apparatus such that the electrically conductive lead defines a portion of the second conductive path. For example, the electrically conductive lead may extend through an opening in the insulating member.

In another embodiment, the disposable may include a set screw that secures the electrically conductive lead to the conductive member. In this regard, the conductive member may be a shaft. Further, the actuation assembly may include a second conductive post secured to the conductive member such that the electrically conductive post defines a portion of the second conductive path.

According to yet another embodiment, the base portion of the bowl may include a conical projection. The electrically non-conductive bowl may be constructed of various materials. For example, the electrically non-conductive bowl may include material selected from the group consisting of quartz glass and borosilicate glass. Further, the electrically conductive apparatus may be constructed of various materials. For example, the electrically conductive apparatus may include material selected from the group consisting of nichrome and iron-chromium-aluminum alloy.

In other instances, the disposable may be included within a system including a control module electrically connected to the actuation assembly. In this regard, manipulation of the control module may transmit an electric current through the actuation assembly to heat the electrically conductive apparatus and the bowl to vaporize the ingestible compound. Further, the disposable may be included within a system including a tube and a hollow adaptor piece fluidically engaged with the tube at an adapter piece outlet. In this regard, the disposable may be disposed within a chamber of the adapter piece and configured to receive the ingestible compound through an adapter piece aperture. In turn, the activation of the electrically conductive apparatus by the control module may heat the electrically non-conductive bowl to vaporize the ingestible compound received in the bowl such that vaporized molecules of the received ingestible compound pass through the adapter piece and into the tube at the adaptor piece outlet.

In this regard, a second aspect of the present disclosure includes a system for vaporizing an ingestible compound. The system includes a tube. The system further includes a hollow adaptor piece fluidically engaged with the tube at an adapter piece outlet. The system further includes a vaporizing assembly disposed within a chamber of the adapter piece and configured to receive an ingestible compound through an adapter piece aperture. In this regard, the vaporizing assembly includes an electrically non-conductive bowl that includes a wall having opposite first and second ends opposite interior and exterior surfaces, an opening adjacent the first end of the wall, a base portion adjacent the second end of the wall, a cavity defined by the base portion and the interior surface of the wall, and an axis extending through the opening, the cavity and the base portion. Notably, the base portion may be closed across a maximum cross-dimension of the cavity. The vaporizing assembly further includes an electrically conductive apparatus disposed over the exterior surface of the wall of the electrically non-conductive bowl and configured for vaporization of the ingestible compound. The system further includes a control module configured to transmit electrical current to the electrically conductive apparatus. In this regard, the transmission of electrical current to the electrically conductive apparatus by the control module may heat the electrically non-conductive bowl to vaporize the ingestible compound at the vaporization assembly such that vaporized molecules of the received ingestible compound pass through the adapter piece and into the tube at the adaptor piece outlet.

A number of feature refinements and additional features are applicable in the second aspect and contemplated in light of the present disclosure. These feature refinements and additional features may be used individually or in any combination. As such, each of the following features that will be discussed may be, but are not required to be, used with any other feature combinations of the second aspect.

For example, the control module may include a depressible actuation button. In this regard, the control module may be configured such that depression of the depressible actuation button activates the electrically conductive apparatus. As another example, in an embodiment, the electrically conductive apparatus may be wrapped over the exterior surface of the wall about the axis. In some instances, the electrically conductive apparatus may be spiral wound over the exterior surface of the wall about the axis. In either event, the electrically conductive apparatus may be a flat wire.

In another embodiment, the vaporizing assembly may further include an actuation assembly electrically interconnected to the electrically conductive apparatus and the control module that electrically energizes the electrically conductive apparatus to heat the electrically non-conductive bowl for vaporizing the ingestible compound. In this regard, the actuation assembly may further include a first electrically conductive path electrically connected to a first end of the electrically conductive apparatus and a second electrically conductive path electrically connected to a second end of the electrically conductive apparatus. Accordingly, the actuation assembly may further include an electrically conductive base member having a first cavity that receives a portion of the electrically non-conductive bowl such that the electrically conductive base member defines a portion of the first conductive path.

Further, the actuation assembly may also include at least one electrically non-conductive fastener receivable through an aperture in the portion of the bowl and an aligned bore in the base member for securing the electrically non-conductive bowl to the base member such that the electrically conductive apparatus is electrically interconnected with the first conductive path of the actuation assembly. In this regard, the bore of the base member may be defined by the electrically conductive inner surface that defines a portion of the first conductive path. In turn, the electrically conductive inner surface may be defined by an electrically conductive insert that may be received within the bore.

In another embodiment, the actuation assembly may further include an electrically conductive spacer member disposed between the portion of the bowl and the base member such that the electrically conductive spacer member defines a portion of the first conductive path. In this regard, the electrically conductive spacer member may be in electrical contact with the first end of the electrically conductive apparatus. In some instances, the actuation assembly may further include a first conductive post that may be received in a second cavity of the base member such that the first conductive post defines a portion of the first conductive path.

In yet another embodiment, the axis of the bowl may extend through the first and second cavities of the base member. In this regard, a longitudinal axis of the at least one fastener may be perpendicular to the axis of the bowl. In some instances, the actuation assembly may further include an insulating member that electrically isolates the first and second conductive paths. As such, the insulating member may be an insulating sleeve disposed about the electrically conductive apparatus. In this regard, a gap may be defined between the insulating member and the electrically non-conductive bowl. For example, the gap may be defined about a substantial entirety of a circumference of the electrically non-conductive bowl.

According to another embodiment, the actuation assembly may further include a conductive member secured to an outside surface of the insulating member opposite the electrically conductive apparatus such that the conductive member defines a portion of the second conductive path. In turn, the actuation assembly may further include an electrically conductive lead electrically connected between the conductive member and the second end of the electrically conductive apparatus such that the electrically conductive lead defines a portion of the second conductive path. For example, the electrically conductive lead may extend through an opening in the insulating member.

In another embodiment, the vaporizing assembly may include a set screw that secures the electrically conductive lead to the conductive member. In this regard, the conductive member may be a shaft. Further, the actuation assembly may include a second conductive post secured to the conductive member such that the electrically conductive post defines a portion of the second conductive path.

According to yet another embodiment, the base portion includes a base surface that tapers from the second end of the wall towards the first end of the wall to distribute the ingestible compound received through the opening onto the base surface towards the interior surface of the wall. For example, the base portion of the bowl may include a conical projection. The electrically non-conductive bowl may be constructed of various materials. For example, the electrically non-conductive bowl may include material selected from the group consisting of quartz glass and borosilicate glass. Further, the electrically conductive apparatus may be constructed of various materials. For example, the electrically conductive apparatus may include a material selected from the group consisting of nichrome and iron-chromium-aluminum alloy.

Further, in another embodiment, the tube may be a water pipe.

In this regard, a third aspect of the present disclosure includes a method for vaporizing an ingestible compound. The method includes placing a mixture of ingestible compounds through an aperture of an adapter piece and onto an interior surface of an electrically non-conductive bowl of a vaporizing assembly disposed within a chamber of the adapter piece. In this regard, the electrically non-conductive bowl includes a wall having opposite first and second ends and opposite interior and exterior surfaces, an opening adjacent the first end of the wall, a base portion adjacent the second end of the wall, a cavity defined by the base portion and the interior surface of the wall, and an axis extending through the opening, the cavity and the base portion. The base portion may include a base surface that tapers from the second end of the wall towards the first end of the wall to distribute an ingestible compound received through the opening onto the base surface towards the interior surface of the wall. Notably, the base surface may be closed across a maximum cross-dimension of the cavity. The method further includes operating a control module to electrically energize an electrically conductive apparatus via an actuation assembly to heat the electrically non-conductive bowl for vaporizing the mixture of ingestible compounds such that vaporized molecules of the ingestible compounds pass through the adapter piece and into a tube fluidically engaged with the adapter piece at an adapter piece outlet. In this regard, the electrically conductive apparatus may be disposed over the exterior surface of the wall of the electrically non-conductive bowl.

A number of feature refinements and additional features are applicable in the third aspect and contemplated in light of the present disclosure. These feature refinements and additional features may be used individually or in any combination. As such, each of the following features that will be discussed may be, but are not required to be, used with any other feature combinations of the third aspect.

For example, the method may further include selectively connecting the vaporizing assembly to the control module. As another example, in an embodiment, the electrically conductive apparatus may be wrapped over the exterior surface of the wall about the axis. In some instances, the electrically conductive apparatus may be spiral wound over the exterior surface of the wall about the axis. In either event, the electrically conductive apparatus may be a flat wire.

In another embodiment, the actuation assembly electrically interconnects with the electrically conductive apparatus and the control module such that operating the control module electrically energizes the electrically conductive apparatus to heat the electrically non-conductive bowl. In this regard, the actuation assembly includes a first electrically conductive path electrically connected to a first end of the electrically conductive apparatus and a second electrically conductive path electrically connected to a second end of the electrically conductive apparatus. In this regard, the actuation assembly may further include an electrically conductive base member having a first cavity that receives a portion of the electrically non-conductive bowl such that the electrically conductive base member defines a portion of the first conductive path.

Further, the actuation assembly may also include at least one electrically non-conductive fastener receivable through an aperture in the portion of the bowl and an aligned bore in the base member for securing the electrically non-conductive bowl to the base member such that the electrically conductive apparatus is electrically interconnected with the first conductive path of the actuation assembly. In this regard, the bore of the base member may be defined by an electrically conductive inner surface that defines a portion of the first conductive path. In turn, the electrically conductive inner surface may be defined by an electrically conductive insert that may be received within the bore.

In another embodiment, the actuation assembly may further include an electrically conductive spacer member disposed between the portion of the bowl and the base member such that the electrically conductive spacer member defines a portion of the first conductive path. In this regard, the electrically conductive spacer member is in electrical contact with the first end of the electrically conductive apparatus. In some instances, the actuation assembly further includes a first conductive post that may be received in a second cavity of the base member such that the first conductive post defines a portion of the first conductive path.

In yet another embodiment, the axis of the bowl may extend through the first and second cavities of the base member. In this regard, a longitudinal axis of the at least one fastener may be perpendicular to the axis of the bowl. In some instances, the actuation assembly may further include an insulating member that electrically isolates the first and second conductive paths. As such, the insulating member may be an insulating sleeve disposed about the electrically conductive apparatus. In that regard, a gap may be defined between the insulating member and the electrically non-conductive bowl. For example, the gap may be defined about a substantial entirety of a circumference of the electrically non-conductive bowl.

According to another embodiment, the actuation assembly may further include a conductive member secured to an outside surface of the insulating member opposite the electrically conductive apparatus such that the conductive member defines a portion of the second conductive path. In turn, the actuation assembly may further include an electrically conductive lead electrically connected between the conductive member and the second end of the electrically conductive apparatus, wherein the electrically conductive lead defines a portion of the second conductive path. For example, the electrically conductive lead may extend through an opening in the insulating member.

In another embodiment, a set screw may secure the electrically conductive lead to the conductive member. In this regard, the conductive member may be a shaft. Further, the actuation assembly may include a second conductive post secured to the conductive member such that the electrically conductive post defines a portion of the second conductive path.

According to yet another embodiment, the base portion of the bowl may include a conical projection. The electrically non-conductive bowl may be constructed of various materials. For example, the electrically non-conductive bowl may include material selected from the group consisting of quartz glass and borosilicate glass. Further, the electrically conductive apparatus may be constructed of various materials. For example, the electrically conductive apparatus may include a material selected from the group consisting of nichrome and iron-chromium-aluminum alloy. Additionally, the operating of the control module may occur before the placing of the mixture of ingestible compounds.

In addition to the exemplary aspects and embodiments described above, further aspects and embodiments will become apparent by reference to the drawings and by study of the following descriptions.

### BRIEF DESCRIPTION OF THE DRAWINGS

FIG. 1A is an isometric view of an embodiment of a disposable vaporizing assembly.

FIG. 1B is a front view of the embodiment of the disposable vaporizing assembly of FIG. 1A.

FIG. 1C is a detailed cross-sectional view of the embodiment of the disposable vaporizing assembly of FIG. 1A taken along the line E-E of FIG. 1B.

FIG. 2 is an isometric view of an embodiment of a disposable vaporizing assembly interconnected with a power transfer assembly for connection with a control module.

FIG. 3A is an isometric view of an embodiment of a disposable vaporizing assembly and interconnected control module.

FIG. 3B is a functional block diagram of a control module and vaporizing assembly for use in a system for vaporizing an ingestible compound, according to one embodiment.

FIG. 4A is an isometric view of an embodiment of a system for vaporizing an ingestible compound.

FIG. 4B is a top view of the embodiment of the system for vaporizing an ingestible compound of FIG. 4A.

FIG. 4C is a detailed cross-sectional view of the embodiment of the system for vaporizing an ingestible compound of FIG. 4B taken along the line D-D of FIG. 4B.

FIG. 5 illustrates with a flow diagram an embodiment of a method for vaporizing an ingestible compound.

### DETAILED DESCRIPTION

Disclosed herein are utilities (e.g., apparatuses, systems, processes, etc.) for vaporizing an ingestible compound (e.g., tobacco, essential oils, herbs, and/or other medicinal or organic materials, etc.) in a manner that is clean, efficient, repeatable, and consistent (e.g., the ingestible compound may vaporize at a repeatably consistent rate across an internal volume of the disclosed utilities, etc.). The disclosed utilities may employ an electrically non-conductive bowl configured to receive a volume of ingestible compounds relative to a tapered base surface opposite an opening of the bowl for distribution of the ingestible compound towards an interior surface of the bowl. In turn, an electrically conductive apparatus disposed over an exterior surface of the bowl may be configured to heat the electrically non-conductive bowl for vaporizing the ingestible compound. For example, an actuation assembly may be electrically interconnected to the electrically conductive apparatus to electrically energize the electrically conductive apparatus to produce heat for use in vaporizing the ingestible compound disposed within the electrically non-conductive bowl.

In some implementations, the disclosed utilities may also include various other components to facilitate the ingestion (e.g., inhalation) of the vaporized compound. For example, the electrically conductive apparatus (and associated actuation assembly) may be electrically interconnected with a control module that is configured to selectively transmit electrical current to the electrically conductive apparatus in response to receiving an indication indicative of the desired initiation of the vaporization of the ingestible compound (e.g., an indication associated with depressing a depressible actuation button at the control module for initiation of the vaporization of the ingestible compound, etc.). As a further example, the electrically non-conductive bowl may be removably housed within a chamber of an adapter piece fluidically engaged with a tube (e.g., a water pipe, etc.) to facilitate the ingestion of the vaporized compound. In this regard, the transmission of electrical current to the conductive apparatus may heat the electrically non-conductive bowl such that vaporized molecules of the received ingestible compound pass through the adapter piece and into the tube to facilitate the ingestion of the vaporized molecules of the vaporized compound.

More broadly, the disclosed embodiments relate to heating an ingestible compound such that it vaporizes (e.g., atomizes) in a clean and efficient manner to facilitate ingestion by a user for various therapies of interest (e.g., aromatherapy, respiratory therapy, smoking, and/or other medicinal or recreational therapies, etc.). For example, the invention may be used to heat an ingestible compound to a temperature at which it undergoes a phase change into a gas free from extraneous (e.g., combustion-produced) chemical reactions (e.g., free from chemical reactions other than the oxidation and/or decomposition of hydrocarbon substances into CO₂ and H₂O). In this regard, a solid-state ingestible compound may undergo a phase change into a gas free from an intermediate combustion step, which may produce a healthier, cleaner vapor.

In this regard, the present invention advances the art of vaporizing ingestible compounds by moving substantially beyond traditional heat-application models (e.g., direct-fire, etc.) to create a whole new approach to vaporing an ingestible compound. For example, in certain embodiments described below, a vaporing assembly is disclosed that electrically actuates an electrically conductive apparatus (e.g., a resistive heating element, etc.) to produce heat for use in vaporizing the ingestible compound. The electrically conductive apparatus and ingestible compound may be separated (i.e., interposed) by an electrically non-conductive bowl (e.g., the electrically conductive apparatus may be disposed adjacent an exterior surface of the bowl, while the ingestible compound may be disposed adjacent an interior surface of the bowl) such that heat produced at the electrically conductive apparatus is thermally conducted through the electrically non-conductive bowl to vaporize the ingestible compound. In this regard, the ingestible compound may be vaporized free from direct contact with the electrically conductive apparatus, thereby facilitating the production of a clean and smooth vapor for use in various therapies of interest.

The invention may also facilitate vaporizing an ingestible compound in a consistent, even, and repeatable manner (e.g., the ingestible compound may vaporize at a consistent rate across an internal volume of the electrically non-conductive bowl, etc.). To facilitate the foregoing, the ingestible compound may be disposed, for example, relative to a base surface of the electrically non-conductive bowl in a manner that facilitates distribution of the ingestible compound towards an interior wall of the bowl (i.e., a portion of the bowl extending between a bowl opening and bowl base). For instance, the base may be closed across a maximum cross-dimension of a cavity of the bowl and be tapered (e.g., via a conical or other geometric protrusion, such as an irregularly shaped protrusion) toward the interior wall of the bowl to support even spreading of the ingestible compound within the cavity of the bowl and thereby facilitate consistent vaporization of the compound (e.g., proportionally spreading the ingestible compound across an internal volume of the bowl relative to the interior wall such that ingestible compound vaporizes at substantially the same rate along a perimeter of the bowl, etc.). The vaporized molecules from the ingestible compound may therefore travel up and out of the opening of the bowl (opposite the base) for ingestion by a user, for example, due in part to the base being closed across a maximum cross-dimension of the cavity of the bowl. For example, the electrically non-conductive bowl may be disposed within a chamber of a hollow adapter piece such that the vaporized molecules travel up and out of the opening, through the hollow adapter piece, and into a tube, whereat the vaporized molecules may be ingested (e.g., via a mouthpiece, hose, and/or any other appropriate implement), during the administration of a given therapy of interest.

It should be appreciated, however, that alternative or additional embodiments of the foregoing utilities may be utilized in accordance with the present invention, and are described in greater detail below. For example, the electrically conductive apparatus may be electrically interconnected with an actuation assembly operable to electrically energize the electrically conductive apparatus via manipulation of an associated control module. The control module may be manipulated via a depressible actuation button and may contain various components configured to transmit electrical current to the heating element and perform any other appropriate functions (e.g., the control module may contain a processing engine, user interface, output, storage module, battery, and/or the like). Moreover, in some implementations, the control module may be programmable to facilitate vaporizing an ingestible compound according to various measured and/or preprogrammed parameters (e.g., according to various properties of the ingestible compound received at the bowl, including compound composition, weight, and/or the like). Accordingly, the description below should be understood as exemplifying particular embodiments and implementations of the invention, and not by way of limitation.

Reference will now be made to the accompanying drawings, which assist in illustrating the various pertinent features of the various novel aspects of the present disclosure. The following description is presented for purposes of illustration and description. Furthermore, the description is not intended to limit the inventive aspects to the forms disclosed herein. Consequently, variations and modifications commensurate with the following teachings, and skill and knowledge of the relevant art, are within the scope of the present inventive aspects.

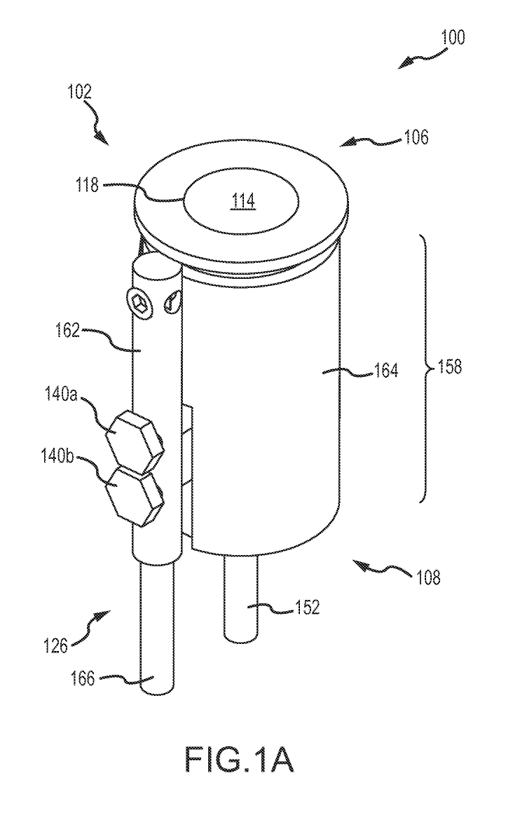

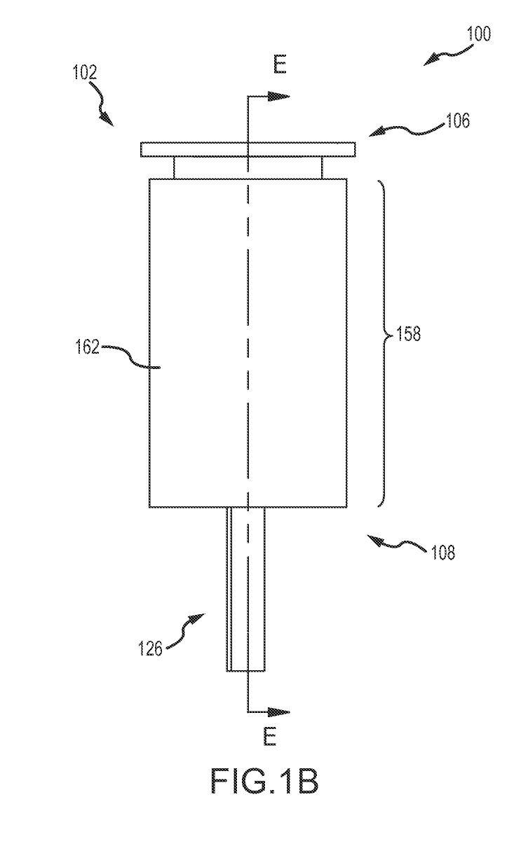

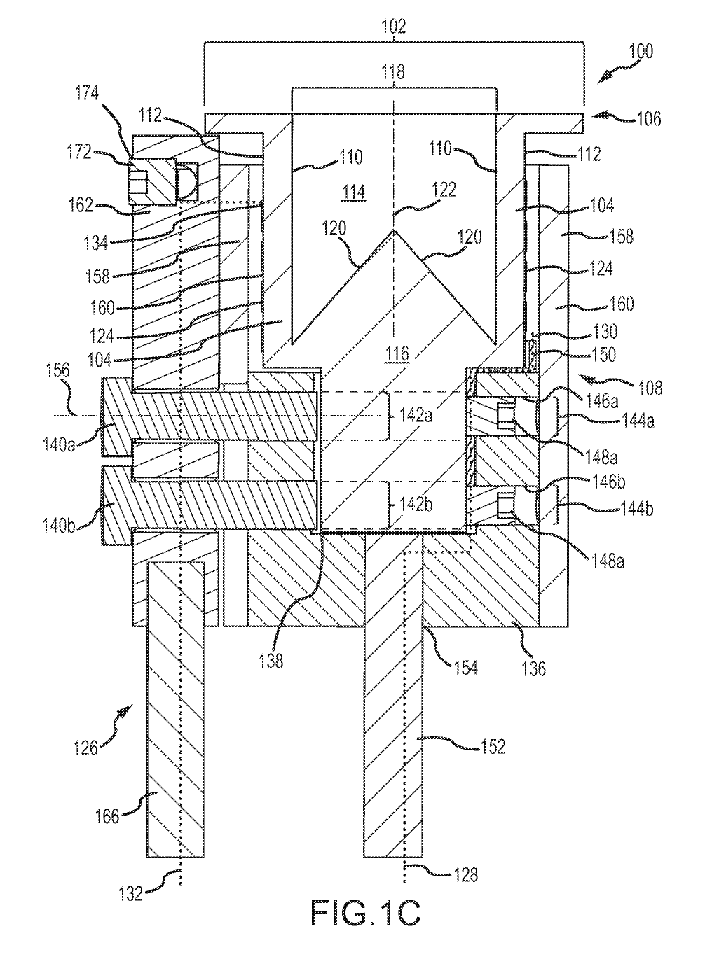

In this regard, FIGS. 1A-1C present an embodiment of a disposable vaporizing assembly 100 for vaporizing an ingestible compound. Broadly, the disposable vaporizing assembly 100 may include an electrically non-conductive bowl 102 configured to receive a volume of ingestible compounds for vaporization (e.g., for atomization of the ingestible compound such that it undergoes a phase change into a gas). The electrically non-conductive bowl 102 may be of any appropriate size and configuration to house the received ingestible compound for vaporization according to the description presented herein. In one embodiment, the electrically non-conductive bowl 102 includes a wall 104 have opposite first and second ends 106, 108. The wall 104 may also include opposite interior and exterior surfaces 110, 112 (i.e., interior and exterior surfaces 110, 112 may be opposing external surfaces of the wall 104). In this regard, the electrically non-conductive bowl 102 may include a cavity 114 defined by the interior surface 110 of the wall 104. The cavity 114 may be further defined by a base portion 116 of the electrically non-conductive bowl 102 disposed adjacent the second end 108 and that is closed across a maximum cross-dimension of the cavity 114. Furthermore, an opening 118 of the electrically non-conductive bowl 102 is disposed adjacent the first end 106. Accordingly, the electrically non-conductive bowl 102 may receive the ingestible compound through the opening 118 such that the ingestible compound is disposed within an internal volume of the cavity 114 in a manner that facilitates vaporization, as described in greater detail below.

In one instance, base portion 116 may be configured in a manner to support the consistent vaporization of the ingestible compound across the internal volume defined by the cavity 114 (e.g., a substantially consistent rate of vaporization corresponding to the rate at which a received volume of solid ingestible compound undergoes a phase change into a gas over a period of time). For example, base portion 116 may include at least one base surface 120 that tapers from the second end 108 towards the first end 106 in any appropriate manner to distribute the ingestible compound received through the opening 118 onto the base surface 120 towards the interior surface 110, which may be heated by a heat source, discussed in greater detail below, disposed adjacent the external surface 112. In this regard, the base portion 116 may automatically distribute ingestible compound received within the electrically non-conductive bowl 102 generally in the direction of the heat source, whereat the temperature in the electrically non-conductive bowl 102 may be greatest and, in turn, may be calibrated to reach a temperature that causes the ingestible compound to vaporize (i.e., the heat source may cause the interior surface 110 to reach a temperature at which the ingestible compound undergoes a phase change into a gas). As such, portions of the ingestible compound proximal to (e.g., against, adjacent, etc.) the interior surface 110 may vaporize before those portions disposed further from the interior surface 110. The base surface 120, may therefore facilitate the consistent vaporization of the ingestible compound by continuously channeling the ingestible compound towards the interior surface 110 until a desired volume is vaporized (i.e., as portions of the ingestible compound proximal to the interior surface 110 are vaporized, the tapered base surface 120 distributes or channels those portions disposed further from the interior surface 110 towards the interior surface 110 for subsequent vaporization until the entirety or a desired volume of the ingestible compound is vaporized).

In some embodiments, the tapered base surface 120 may be substantially symmetrical about an axis 122 extending through the opening 118, the cavity 114, and the base portion 116. In this regard, the tapered base surface 120 may distribute the ingestible compound substantially evenly in relation to the interior surface 110 to support the consistent vaporization of the ingestible compound (i.e., a predetermined volume of ingestible compound may be continuously disposed proximal the interior surface 110 while the electrically non-conductive bowl is heated for vaporization of the ingestible compound). Additionally or alternatively, the tapered base surface 120 may be defined by a conical or other geometric protrusion extending from the base portion 116 towards the first end 106, as may be appropriate for the geometry of the electrically non-conductive bowl 102. For example, in one embodiment, the cavity 114 may be substantially cylindrical. In this regard, a tapered base surface 120 defined by a conical protrusion disposed symmetrically about axis 122 may support the distribution of ingestible compounds towards the interior surface 110. In certain other embodiments, the tapered base surface 120 may be defined by a bulbous, spherical (convex or concave) or other geometric protrusion extending from the base portion 116 towards the first end 106, including configurations of the tapered base surface 120 not symmetrical with respect to the axis 122 (e.g., a tapered base surface 120 configured to distribute ingestible compound towards the interior surface 110 of an irregularly shaped electrically non-conductive bowl 102, etc.). In other cases, different protrusion geometries may be appropriate to facilitate the distribution of the ingestible compound towards the interior surface 110.

The electrically non-conductive bowl 102 may be constructed of any appropriate material that functions as an electrical insulator (i.e., a material in which electric charges do not flow freely such that an electric current may not be passed through the material). In one embodiment, the electrically non-conductive bowl 102 may be constructed from quartz glass. In another embodiment, the electrically non-conductive bowl 102 may be constructed from borosilicate glass or another analogously appropriate material. In any event, electrical current may be prevented from flowing through the electrically non-conductive bowl 102. In this regard, with such construction, the interior surface 110 may be free from electrical charge (i.e., current may not flow between any two points at the interior surface 110), even where an electrically charged element is disposed adjacent exterior surface 112 (e.g., as in the case of a resistive heating element, etc.). As such, an ingestible compound received relative to the interior surface 110 may be vaporized free from contact with an electrically charged surface, which may thereby promote a smooth and clean vaporization (e.g., free from combustion, etc.) of the ingestible compound, according to the embodiments described herein.

Further, as shown in FIG. 1C, the base surface 120 may be closed across a maximum cross-dimension of the cavity 114. In this regard, the wall 104 and base portion 116 define a solid, closed barrier of the cavity 114. As such, as the ingestible compound is vaporized within the electrically non-conductive bowl 102, vaporized gas molecules of the ingestible compound may exit the cavity 114 through the opening 118. That is, the vaporized gas molecules may exit the cavity 114 through the same opening 118 through which the ingestible compound was received for at least because the vaporized gas molecules may not pass through the solid barrier of the wall 104 or base portion 120.

The electrically non-conductive bowl 102 may be externally heated in order to vaporize the ingestible compounds received within the cavity 114. In this regard, the disposable vaporizing assembly 100 may include an electrically conductive apparatus 124 disposed over the exterior surface 112 of the electrically non-conductive bowl 102. Accordingly, the ingestible compound may be vaporized free from direct contact with the electrically conductive apparatus 124.

In one embodiment, the electrically conductive apparatus 124 may be a resistive heating element. In this regard, electric current passed through the electrically conductive apparatus 124 may encounter resistance configured to cause the electrically conductive apparatus 124 to release a predetermined amount of heat. In turn, the heat released at the electrically conductive apparatus 124 may heat the electrically non-conductive bowl 102 to a temperature sufficient to cause vaporization of the ingestible compound contained therein. In some cases, the heat released at the electrically conductive apparatus 124 may cause the electrically non-conductive bowl 102 to reach a temperature of at least 700° Fahrenheit. In other cases, the heat released at the electrically conductive apparatus 124 may cause the electrically non-conductive bowl 102 to reach a temperature of more or less than 700° Fahrenheit, as may be appropriate for a given application (e.g., various different ingestible compounds may each vaporize at different temperatures, etc.). For example, the electrically conductive apparatus 124 may cause the electrically non-conductive bowl 102 to reach a temperature of at least 650° Fahrenheit, or at least 600° Fahrenheit, or at least any other appropriate temperature. In some embodiments, the electrically conductive apparatus 124 may be configured to cause the electrically non-conductive bowl 102 to reach a predefined temperature at least partially based on the composition of the ingestible compound (e.g., the electrically conductive apparatus 124 may emit a predetermined amount of heat in relation to a received indication indicative of an identified material composition, for example, as identified at a sensor of an interconnected control module, etc.).

The electrically conductive apparatus 124 may be of any appropriate configuration (e.g., material composition, geometric properties, disposition relative to the electrically non-conductive bowl 102, etc.) to facilitate heating the electrically non-conductive bowl 102 for vaporization of the ingestible compound. In this regard, the electrically conductive apparatus 124 may be configured such that passing a predefined level of current through the electrically conductive apparatus 124 causes the electrically non-conductive bowl 102 to reach a particular temperature. For example, in an embodiment in which the electrically non-conductive bowl 102 reaches 700° Fahrenheit, a first configuration of the electrically conductive apparatus 124 may be appropriate. As a further example, in an embodiment in which the electrically non-conductive bowl 102 reaches a temperature greater or less than 700° Fahrenheit, a second configuration of the electrically conductive apparatus 124 may be appropriate. That is, the material composition, geometric properties, and disposition (relative to the electrically non-conductive bowl 102) of the electrically conductive apparatus 124 may be tailored such that a desired amount of heat is emitted from the electrically conductive apparatus 124 to heat the electrically non-conductive bowl 102 to a desired temperature.

For example, the material composition of the electrically conductive apparatus 124 may influence the temperature to which the electrically non-conductive bowl 102 is heated (e.g., various different materials may have different resistive properties, which may influence the quantity of heat generated at the electrically conductive apparatus 124 upon the application of current therethrough, etc.). In this regard, in one embodiment, the electrically conductive apparatus 124 may be constructed from nichrome. In another embodiment, the electrically conductive apparatus 124 may be constructed from an iron-chromium-aluminum alloy (e.g., such as Kanthal®) or any other analogously appropriate material. In any event, a particular conductive material may be selected such that the electrically conductive apparatus 124 generates heat that causes the electrically non-conductive bowl 102 to reach the appropriate temperature for a given application.

Further, the geometry (e.g., cross-sectional dimensions of the electrically conductive apparatus 124) and the disposition (e.g., disposition relative to the electrically non-conductive bowl 102) of the electrically conductive apparatus 124 may facilitate heating the electrically non-conductive bowl 102 to a desired temperature. For example, the geometry and disposition of the electrically conductive apparatus 124 may influence the rate at which heat is transferred from the electrically conductive apparatus 124 to the electrically non-conductive bowl 102. In one embodiment, the electrically conductive apparatus 124 may be wrapped over the exterior surface 112 of the electrically non-conductive bowl 102. In this regard, the electrically conductive apparatus 124 may be in direct contactable relation with the exterior surface 112 to facilitate heating the electrically non-conductive bowl 102 through the transfer of heat via the surface contact of the electrically conductive apparatus 124 with the exterior surface 112.

Additionally, and as depicted in FIG. 1C, the electrically conductive apparatus may be spiral wound over the exterior surface 112 about the axis 122. For instance, the electrically conductive apparatus 124 may be wrapped over at least half the distance between the first and second ends 106, 108, such as at least 75% between the first and second ends 106, 108 to facilitate even heat distribution within the electrically non-conductive bowl 124 (i.e., the electrically non-conductive bowl 102 may receive heat from the electrically conductive apparatus 124 throughout the surface area of the exterior surface 112, which may result in a consistent or even temperature at the wall 104).

Further, the electrically conductive apparatus 124 may be a flat wire. The flat wire may increase the efficiency of the heat transferred between the electrically conductive apparatus 124 and the electrically non-conductive bowl 102 by increasing the surface area of the electrically conductive apparatus 124 in direct contactable relation with the electrically non-conduction bowl 102. In this regard, it will be appreciated that any manner of the foregoing materials, geometries, or dispositions of the electrically conductive apparatus 124, taken individually or in any appropriate combination, may be used to cause the electrically non-conductive bowl 102 to be heated to a desired temperature (i.e., the particular materials, geometries, and dispositions of the electrically conductive apparatus 124 may be tailored such that, upon the application of current, the electrically conductive apparatus 124 emits heat at a level sufficient to heat the electrically non-conductive bowl 102 to a temperature that may vaporize the ingestible compound).

In one arrangement, the electrically non-conductive bowl 102 may be heated by the electrically conductive apparatus 124 through various heat transfer mechanisms. For example, heat produced at the electrically conductive apparatus 124 may be transferred to the electrically non-conductive bowl 124 due in part to the contactable disposition of the electrically conductive apparatus 124 in relation to the exterior surface 112 of the electrically non-conductive bowl 102 (i.e., heat produced at the electrically conductive apparatus 124 may pass from one surface of the electrically conductive apparatus 124 to the exterior surface 112 of the electrically non-conductive bowl 102 substantially free from any intervening layer or barrier). The electrically conductive apparatus 124 may also produce heat that may cause the environment in which the electrically non-conductive bowl 102 is disposed to increase in temperature. That is, the electrically conductive apparatus 124 may emit heat from a surface that does not directly contact the electrically non-conductive bowl 102 so as to radiatively heat the electrically non-conductive bowl 102. In this regard, the electrically non-conductive bowl 102 may be heated over and throughout the exterior surface 112, including portions of the exterior surface 112 that may not be in direct mechanical contact (i.e., adjacent to) with the electrically conductive apparatus 124.

The electrically conductive apparatus 124 may be electrically interconnected to an actuation assembly 126 that is configured to electrically energize the electrically conductive apparatus 124 for generation of heat by the electrically non-conductive bowl 124. In this regard, the actuation assembly 126 may supply electrical current to the electrically conductive apparatus 124 in order to produce heat at the electrically conductive apparatus 124 for heating the electrically non-conductive bowl 102 for vaporization of the ingestible compounds. The actuation assembly 126 may include a first electrically conductive path 128 electrically connected to a first end 130 of the electrically conductive apparatus 124 and a second electrically conductive path 132 electrically connected to a second end 134 of the electrically conductive apparatus 124. As such, current may travel from the first end 130 to the second end 134 (or from the second end 134 to the first end 130) via the electrical connection with the respective first electrically conductive path and second electrically conductive path 128, 132.

The actuation assembly 126 may include various structural components in order to supply the electrically conductive apparatus 124 with an appropriate flow of electrical current. That is, the first and second electrically conductive paths 128, 132 may be defined by, or travel through, various components of the disposable vaporizing assembly 100 in order to form a complete circuit that includes the electrically conductive apparatus 124. In one arrangement, the actuation assembly 126 may include an electrically conductive base member 136 (e.g., constructed at least in part from nichrome, an iron-chromium-aluminum alloy, or any other electrically conductive material or combinations thereof, etc.) that defines a portion of the first conductive path 128 and that is configured to interconnect with the electrically non-conductive bowl 102 in any appropriate manner. For instance, the electrically conductive base member 136 may include a first cavity 138 for receiving a portion of the electrically non-conductive bowl 102 (e.g., such as a corresponding protrusion of the base portion, not labeled). Further, the actuation assembly 126 may include at least one electrically non-conductive fastener 140a receivable through an aperture 142a in the portion of the electrically non-conductive bowl 102 received by the electrically conductive base 136 and at least one aligned bore 144a in the electrically conductive base member 136. In this regard, the at least one electrically non-conductive fastener 140a may facilitate the securement of the electrically non-conductive bowl 102 to the electrically conductive base member 136 such that the electrically conductive apparatus 126 may be electrically interconnected with the first conductive path 128.

Accordingly, the at least one electrically non-conductive fastener 140a may mechanically secure the electrically non-conductive bowl 102 to the electrically conductive base 136 to establish a connection at the first end 130 through which the current of first electrically conductive path 128 may flow to (or from) the electrically conductive apparatus 124 to produce heat for use in vaporizing the ingestible compound. In some embodiments, as shown in FIG. 1C, the actuation assembly 126 may include a second electrically non-conductive fastener 140b configured to similarly facilitate the securement of the electrically non-conductive bowl 102 to the electrically conductive base member 136 such that the electrically conductive apparatus 126 may be electrically interconnected with the first conductive path 128. Accordingly, the second electrically non-conductive fastener 140b may be receivable through a second aperture 142b in the in the portion of the electrically non-conductive bowl 102 received by the electrically conductive base member 136 and a second aligned bore 144b in the electrically conductive base member 136.

In one arrangement, the first electrically conductive path 128 may be established in part by an electrically conductive inner surface 146a of the aligned bore 144a that defines a portion of the first conductive path 128. That is, current may flow through the electrically conductive base member 136 along the first electrically conductive path 128 via the conductive inner surface 146a. Similarly, current may also flow through the electrically conductive base member 136 along the first electrically conductive path 128 via a conductive inner surface 146b of the second aligned bore 144b. For instance, the electrically conductive inner surfaces 146a, 146b may be defined by a corresponding electrically conductive insert 148a, 148b that is received within the corresponding aligned bore 144a, 144b. In this regard, current of the first electrically conductive path 128 may flow through the electrically conductive base member 128 via the one or both of the electrically conductive inserts 148a, 148b.

The first electrically conductive path 128 may be further established in part by an electrically conductive spacer member 150 disposed between the electrically non-conductive bowl 102 and the electrically conductive base member 136. For example, the electrically conductive spacer member 150 may be at least partially disposed within the cavity 138 between the electrically conductive base member 136 and the portion of the electrically non-conductive bowl 102 received by the electrically conductive base 136, as shown in FIG. 1C. In this regard, the electrically conductive spacer member 150 may establish an electrical connection between the electrically conductive base 136 and the first end 130 of the electrically conductive apparatus 124 such that current may flow through the first electrically conductive path 128 to (or from) the electrically conductive apparatus 124.

The actuation assembly 126 may also facilitate connecting the vaporizing assembly 100 to an external power source that provides the power (e.g., a supply of current) for electrically energizing the electrically conductive apparatus 124. For example, the actuation assembly 126 may be electrically interconnected with a control module or other power source, discussed in greater detail below, which may provide the appropriate flow of current to the electrically conductive apparatus 124 to heat the electrically non-conductive bowl 102 to the desired temperature for vaporization of the ingestible compound. In part to facilitate such connection to an external power source, the first conductive path of the actuation assembly 126 may also include or be defined by a first conductive post 152 that may be received in a second cavity 154 of the electrically conductive base member 136. In this regard, the first conductive post 152 may define a portion of the first conductive path 128. As such, according to one embodiment, the first electrically conductive path 128 may allow current to flow between the first conductive post 152 and the electrically conductive apparatus 124 via the electrically conductive base member 136 and the electrically conductive spacer member 150. In other implementations, more or fewer components may define the first electrically conductive path 128.

In one arrangement, the first cavity 138 may be aligned relative to the second cavity 154. For example, the first cavity 138 may be aligned relative to the second cavity 154 such that the axis 122 of the electrically conductive bowl 102 extends through the first and second cavities 138, 154 of the electrically conductive base member 136. The securement of the electrically conductive apparatus 124 to the electrically conductive base member 136 may be further facilitated by aligning the at least one electrically non-conductive fastener 142a in relation to the electrically non-conductive bowl 102 in any appropriate manner that supports the mechanical attachment of the electrically non-conductive bowl 102 to the electrically conductive base member 136. For example, in one implementation, the at least one electrically non-conductive fastener 142a may be disposed such that a longitudinal axis 156 of the at least one electrically non-conductive fastener may be perpendicular to the axis 122 of the electrically non-conductive bowl 102.

The electrically non-conductive bowl 102 may be heated by the electrically conductive apparatus 124 through various heat transfer mechanisms. For example, heat produced at the electrically conductive apparatus 124 may be transferred to the electrically non-conductive bowl 124 due in part to the contactable disposition of the electrically conductive apparatus 124 in relation to the exterior surface 112 of the electrically non-conductive bowl 102 (i.e., heat produced at the electrically conductive apparatus 124 may pass from one surface of the electrically conductive apparatus 124 to the exterior surface 112 of the electrically non-conductive bowl 102 substantially free from any intervening layer or barrier). The electrically conductive apparatus 124 may also produce heat that may cause the environment in which the electrically non-conductive bowl 102 is disposed to increase in temperature. That is, the electrically conductive apparatus 124 may emit heat at a surface that may not directly contact the electrically non-conductive bowl 102 so as to heat an environment exterior to the electrically non-conductive bowl 102. In this regard, the electrically non-conductive bowl 102 may be heated over and throughout the exterior surface 112, including portions of the exterior surface 112 that may not be in direct mechanical contact (i.e., adjacent to) with the electrically conductive apparatus 124.

Various components of the actuation assembly 126 may facilitate heating the electrically non-conductive bowl 102 via the emission of heat into the exterior environment of the electrically non-conductive bowl 102 (i.e., exterior to the wall 104) via the electrically conductive apparatus 124. For example, the actuation assembly 126 may include an insulating member 158 configured in part to thermally and electrically insulate the electrically non-conductive bowl 102. In this regard, in one embodiment, the insulating member 158 may be in spaced relation along at least a portion of exterior surface 112 to define a gap 160 such that heat produced at the electrically conductive apparatus 124 may be captured between the insulating member 158 and the electrically non-conductive bowl 102 to facilitate increasing the temperature of the electrically non-conductive bowl 102. In other instances, the insulating member 158 may be disposed such that the gap 160 is defined about a substantial entirety of the circumference of the electrically non-conductive bowl 102.

Additionally, the insulating member 158 may electrically isolate or separate the first and second electrically conductive paths 128, 132 such that the first and second electrically conductive paths 128, 132 may be electrically interconnected by the electrically conductive apparatus 124 (e.g., by connecting the first electrically conductive path 128 to the first end 130 and the second electrically conductive path 132 to the second end 134, etc.) for the production of heat thereat. In this regard, the insulating member 158 may be disposed about the electrically conductive apparatus 124 such that the first and second electrically conductive paths 128, 132 may be electrically isolated while allowing current to flow between the first and second electrically conductive paths 128, 132 via the electrically conductive apparatus 124.

The second electrically conductive path 132 of the actuation assembly 126 may include various components and structures that allow current to flow therethrough to the electrically conductive apparatus 124. For example, the actuation assembly 126 may include a conductive member 162 (e.g., a post, bracket, etc.) secured in any appropriate manner to an outside surface 164 of the insulating member 158 opposite the electrically conductive apparatus 124. For instance the fasteners 140a, 140b may secure the conductive member 162 to the outside surface 164 of the insulating member 158. In this regard, the conductive member 162 may define a portion of the second electrically conductive path 132 such that electric current may flow through the second electrically conductive path 132 at least partially via the conductive member 162. Additionally or alternatively, the actuation assembly 126 may also include a second conductive post 166 secured to the conductive member 162, which may define another portion of the second electrically conductive path 132. In this regard, according to one embodiment, current may flow through the second electrically conductive path 132 via the conductive member 162 and/or the second conductive post 166.

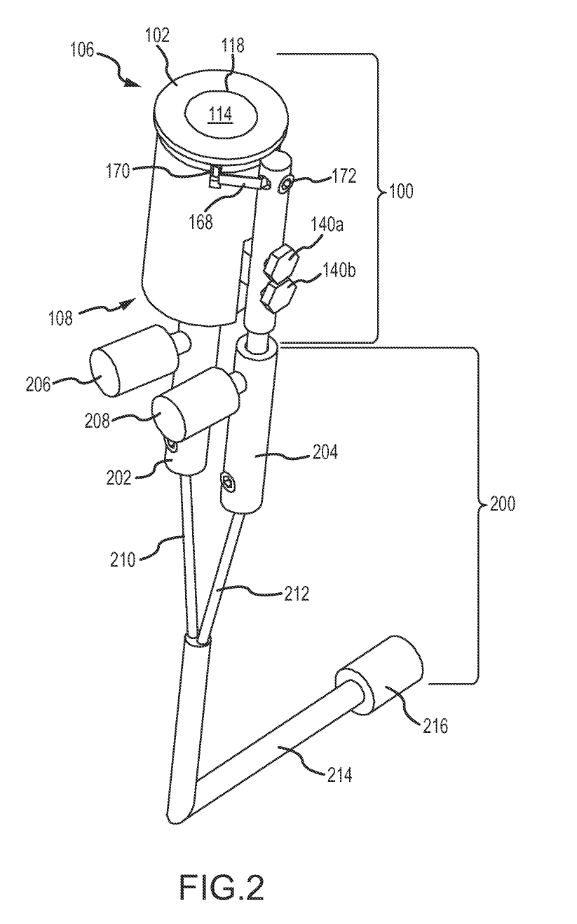

The actuation assembly 126 may facilitate the electrical interconnection of the second electrically conductive path 132 with the electrically conductive apparatus 124. That is, the actuation assembly 126 may include one or more components that facilitate the flow of electrical current between the second electrically conductive path 132 and the electrically conductive apparatus 124, notwithstanding the insulating member 158, which electrically isolates the first and second electrically conductive paths 128, 132. In one arrangement, the actuation assembly 126 may include an electrically conductive lead 168 (best shown in FIG. 2) electrically interconnected between the conductive member 162 and the second end 134 of the electrically conductive apparatus 124 in any appropriate manner. Accordingly, the electrically conductive lead 168 may define another portion of the second electrically conductive path 132 such that current may flow through the second electrically conductive path 132 to/from the electrically conductive apparatus 124.

As discussed previously, the conductive member 162 and the electrically conductive apparatus 124 may be interposed by the insulating member 158. In this regard, the electrically conductive lead 168 may extend through an opening 170 (best shown in FIG. 2) disposed through the insulating member 158 and contact the second end 134 of the electrically conductive apparatus 124 to electrically interconnect the second electrically conductive path 132 with the electrically conductive apparatus 124. In some implementations, a set screw 172 may secure the electrically conductive lead 168 to the conductive member 162. The set screw 172 may be disposed in an aperture 174 of the conductive member 162 proximal to the first end 106. In this regard, the electrically conductive lead 168 may extend generally from the aperture 174 and through the opening 170 to contact the second end 134 of the electrically conductive apparatus 124. In one arrangement, the electrically conductive lead 168 may be an extension of the electrically conductive apparatus 124 such that the electrically conductive lead 168 may be integral with the electrically conductive apparatus 124 to constitute a single continuous segment of wire (e.g., a nichrome, or other appropriately electrically conductive material, wire extending continuously from the electrically conductive apparatus 124 and through the opening 170 for connection with the conductive member 162). Alternatively, the electrically conductive lead 168 may be a leaf spring configured to apply a biasing force against the second end 134 of the electrically conductive apparatus 124, thereby facilitating the electrical connection between the electrically conductive lead 168 and the electrically conductive apparatus 124.

Turning next to FIG. 2, the disposable vaporizing assembly 100 is depicted with power transfer assembly 200. Power transfer assembly 200 may be operable to electrically interconnect the disposable vaporizing assembly 100 with any appropriate control module, power source, and/or other device that allows current to flow through the first and second electrically conductive paths 128, 132 to heat the electrically conductive apparatus 124. In this regard, the power transfer assembly may include first and second terminal receivers 202, 204 configured to electrically interconnect the power transfer assembly 200 with the first and second electrically conductive paths 128, 132, respectively. In one implementation, the first and second terminal receivers 202, 204 may be configured to receive the first conductive post 152 and second conductive post 166, respectively. In one arrangement, the first conductive post 152 and second conductive post 166 may be secured at the first and second terminal receivers 202, 204 via first and second terminal knobs 206, 208. For example, the disposable vaporizing assembly 100 may be advanced into the first and second terminal receivers 202, 204 and secured relative to the power transfer assembly 200 by manipulating the first and second terminal knobs 206, 208 (e.g., by rotating clock-wise or counter clock-wise, etc.). In this regard, the power transfer assembly 200 may be configured to receive any one of a plurality of disposable vaporizing assemblies 100. That is, the vaporizing assembly 100 and the power transfer assembly 200 may be seperably connected at the first and second terminal receivers 202, 204 such that various other vaporizing assemblies 100 may be subsequently interconnected with the power transfer assembly 200 (e.g., in order to replace a vaporizing assembly 100 in a system for vaporizing an ingestible compound, as described in greater detail below).

The power transfer assembly 200 may include various conductive elements, switches, and related circuitry to facilitate provision of electrical current to the disposable vaporizing assembly 100. In this regard, the power transfer assembly 200 may include first and second cables 210, 212 electrically interconnected with the first and second terminal receivers 202, 204. The first and second cables 210, 212 may be electrically conductive such that current may flow between the vaporizing assembly 100 and, for example, an electrically interconnected control module. In this regard, the first and second cables 210, 212 may form another portion of the first and second electrically conductive paths 128, 132, respectively. The first and second cables 210, 212 may, in one embodiment, be disposed within a conduit 214, as shown in FIG. 2. The conduit 214 (and corresponding cables 210, 212 disposed therein) may span any appropriate length until ultimately terminating at a mating piece 216 configured for interconnection with a control module and/or other power source. In this regard, the mating piece 216 may include any appropriate circuitry, switches, and/or the like to facilitate connecting the conduit 214 to such control module.

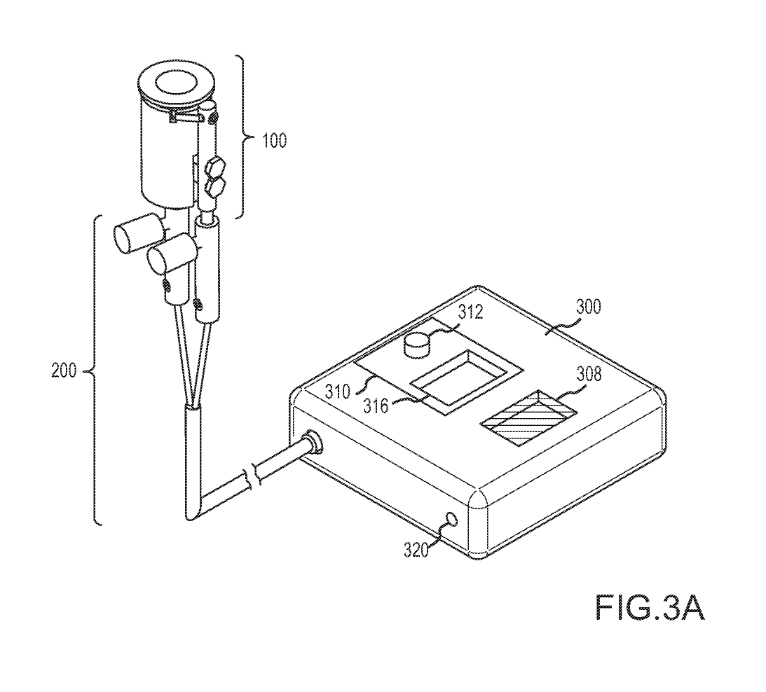

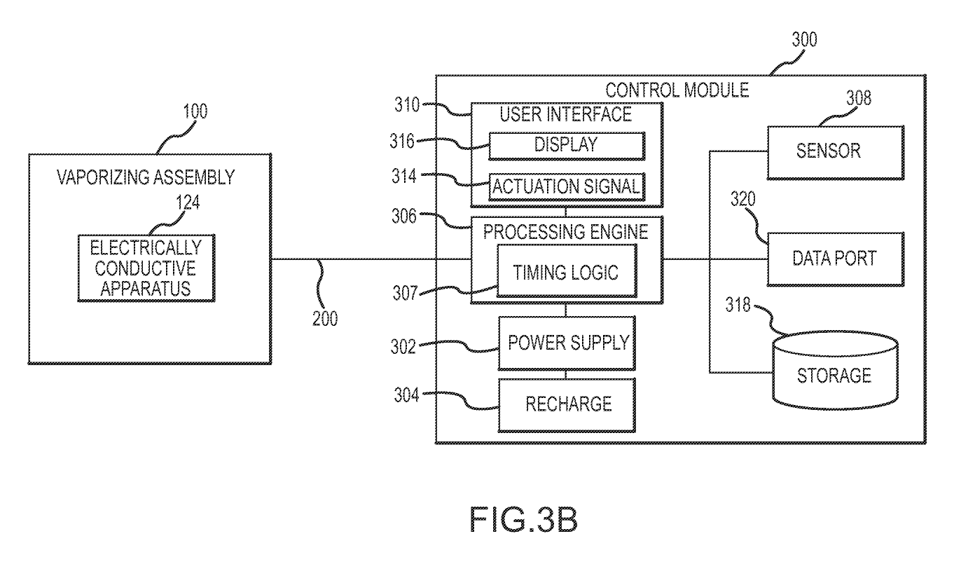

Turning next to FIGS. 3A-3B, a control module 300 is depicted interconnected with the vaporizing assembly 100 via the power transfer assembly 200. The control module 300 may perform any appropriate function to facilitate the production of heat at the electrically conductive apparatus 124. For example, the control module 300 may cause current to flow through the electrically conductive apparatus 124 via the interconnected first and second electrically conductive paths 128, 132 and the power transfer assembly 200. In this regard, the control module may include power supply module 302 (e.g., a battery, or other voltage differential, etc.) that facilitates the flow of current to and from the electrically conductive apparatus 124. The power supply module 302 may be recharged and/or energized by a recharge module 304, which may include a re-charge induction coil and/or a port (not pictured) for connecting to an AC power source or other recharging power source. As such, the control module 300 may transmit current to the electrically conductive apparatus 124 based at least on part on energy stored within, or otherwise accessed from, the control module 300.

In one embodiment, the control module 300 may be configured to selectively transmit current to the electrically conductive apparatus 124 based in part on a received indication (e.g., an indication from a user, a determined or preprogrammed indication from a processor, etc.). In this regard, in response to receiving an indication at the control module 300, current may be transmitted to the electrically conductive apparatus 124 to heat the electrically non-conductive bowl 102 for vaporization of the ingestible compound.

In this regard, to facilitate the foregoing, the control module 300 may include various components (e.g., one or more engines, modules, sensors, antennas, and the like) to facilitate the transmission of current to the electrically conductive apparatus 124 and/or perform any other appropriate functions (e.g., such as collecting, storing, and transmitting data associated with the operation of the control module 300, as explained in greater detail below). For example, as depicted according to the embodiment of FIG. 3B, the control module may include a processing engine 306 (e.g., one or more processors, processing engines, CPUs, etc.) that may be configured to execute one or more logic routines or programs (e.g., such as timing logic 307, etc.) in relation to an indication received at the control module 300. In this regard, the control module 300 may include a user interface 310 and a sensor 308 interconnected with the processing engine 306 and configured to receive an indication for use in determining an appropriate time interval (e.g., time of initiation, duration, etc.) for transmission of current to the electrically conductive apparatus 124. That is, the control module 300 may be configured to transmit current to the electrically conductive apparatus 124 based on a received indication at the control module 300.

In this regard, the user interface 310 may be disposed at an exterior surface of the control module 300 and configured to receive an indication from a user and/or present information to a user in relation to the electrical activation of the electrically conductive apparatus 124 by the control module 300. For example, in one embodiment, the user interface 310 may include depressible actuation button 312. Depressible actuation button 312 may be configured to receive an indication from a user (e.g., by depression of the depressible actuation button) that may cause the control module 300 to pass electrical current through the electrically conductive apparatus 124. For example, upon receiving an indication that depressible actuation button 312 is depressed, the control module 300 may generate actuation signal 314 at the user interface 310 for transmission to and analysis by processing engine 306 (e.g., a preprogrammed logical module, such as timing logic 307, etc.). In this regard, the processing engine 314 may cause current to be transmitted to the electrically conductive apparatus 124 based on the received indication of the depressible actuation button 312 at the user interface 310. The user interface 310 may also include display 316 (e.g., LCD screen, LED screen, or the like) that presents information in relation to the transmission of current to the electrically conductive apparatus 124 or any other appropriate information in connection with the operation of the vaporizing assembly 100. For example, the display 316 may indicate the duration for which current may be transmitted to the electrically conductive apparatus 124.

Additionally or alternatively, the processing engine 314 may cause current to be transmitted to the electrically conductive apparatus 124 based in part on an indication received at the sensor 308, which may facilitate real-time or dynamic control of the vaporization of ingestible compounds at the electrically non-conductive bowl 102. For example, the sensor 308 may be configured to determine one or more parameters of interests (e.g., including compound material composition, weight, density, and the like) in relation to the ingestible compound received at the electrically non-conductive bowl 102. These parameters of interest, in turn, may facilitate the manner in which the control module 300 electrically energizes the electrically conductive apparatus 124. For example, the control module 300 may electrically charge the electrically conductive apparatus 124 for a determined duration and/or allow a certain current flow to be transmitted based on one or more of the measured parameters of interest. For example, it may be desired to electrically charge the electrically conductive apparatus 124 based on the material composition of the ingestible compound, as indicated by the sensor 308.

To facilitate the foregoing, the processing engine 306 may access storage 318 (e.g., one or more magnetic disks, solid-state drives, or other non-volatile memory modules) to facilitate the transmission of current to the electrically conductive apparatus 124 based in part on one or more indications received from the sensor 308. For example, an indication received at sensor 308 may be compared with corresponding data, metrics, or other relevant criteria to determine one or more parameters for transmitting current to the electrically conductive apparatus 124 (e.g., the duration of the transmission of current, etc.). In some embodiments, control module 300 may include data port 320 (e.g., a USB port) configured to receive and transmit information associated with the operation of the control module 300 (e.g., to upload information to storage 318, etc.).

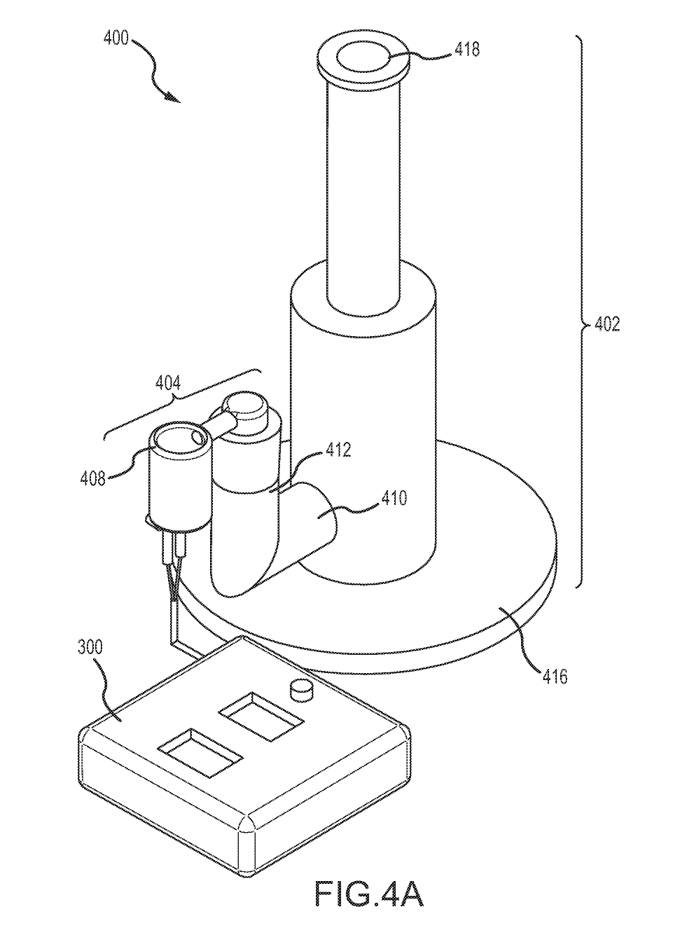

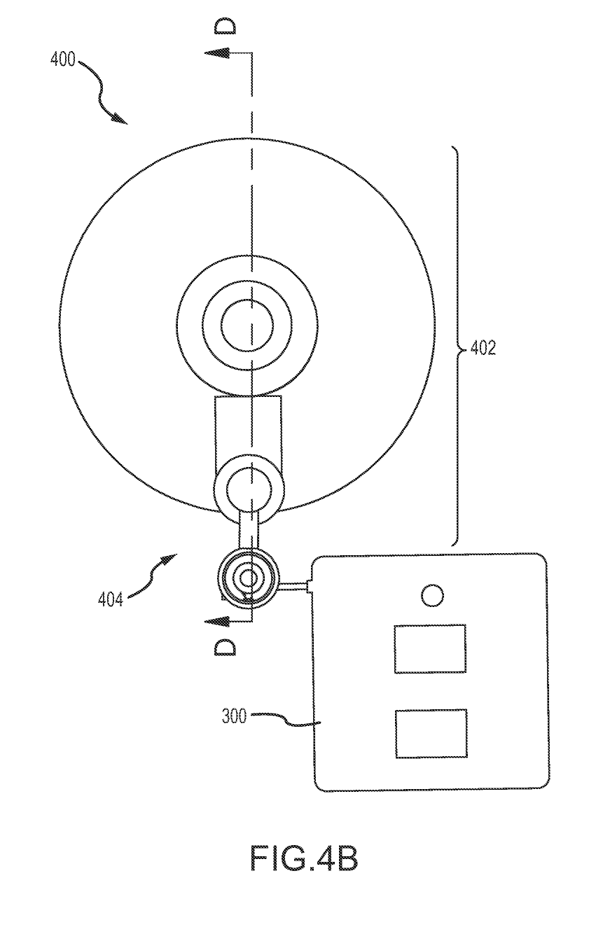

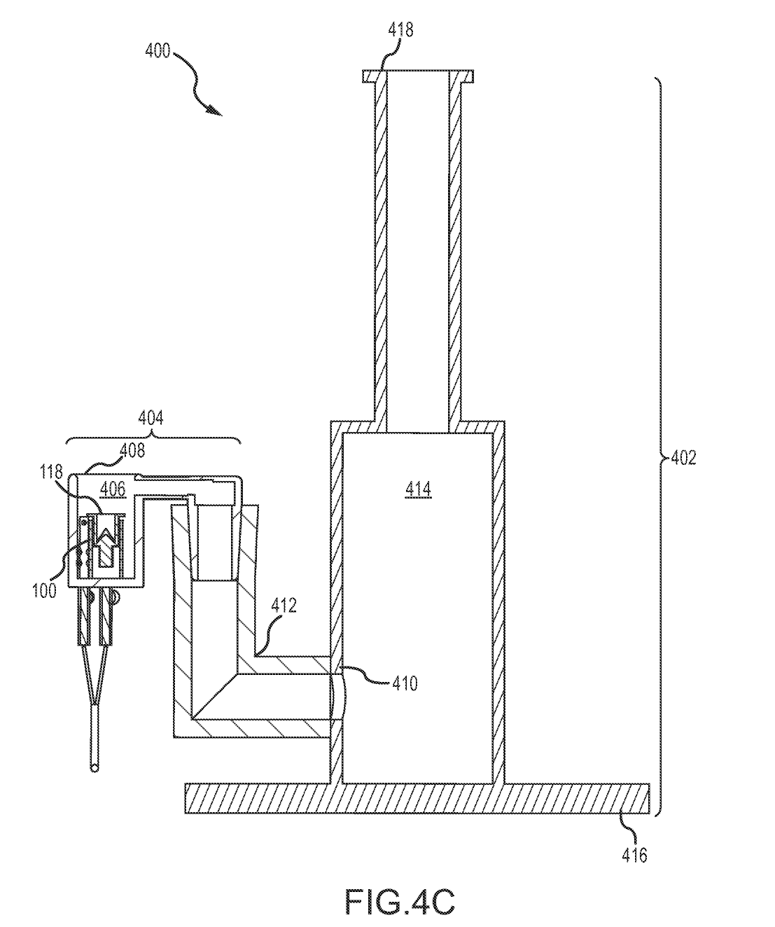

Turning next to FIGS. 4A-4C, an embodiment of a system 400 for vaporizing an ingestible compound is depicted. The system 400 may include any appropriate components to facilitate vaporizing an ingestible compound and ingesting the resulting vaporized molecules. In this regard, the system 400 may include the vaporizing assembly 100, the power transfer assembly 200, and the control module 300 according to the embodiments described in relation to FIGS. 1A-3B. The assembly 400 may also include a tube 402 (e.g., a water pipe and/or other implements designed to facilitate ingestion of the vaporized molecules, etc.) and a hollow adapter piece 404 to facilitate ingestion of the vaporized molecules of the ingestible compound.

In this regard, according to one implementation, the vaporizing assembly 100 may be disposed relative to the hollow adapter piece 404 to allow vaporized molecules to travel through the tube 402 for ingestion by a user. For example, as shown best in FIG. 4C, the vaporizing assembly 100 may be disposed with a chamber 406 of the hollow adapter piece 404. In this regard, the vaporizing assembly 100 may be configured to receive ingestible compounds (i.e., at the electrically non-conductive bowl 102) through an adapter piece aperture 408. The electrically non-conductive bowl 102 may be disposed within the chamber 406 such that the adapter piece aperture 408 is aligned with the electrically non-conductive bowl 102 about the axis 122. In one arrangement, the adapter piece aperture 408 includes a cross-dimension substantially the same as the cross-dimension of the opening 118 of the electrically non-conductive bowl 102. In turn, the adapter piece aperture 408 may allow for a clean vaporization of the ingestible compound by allowing a desired flow of air through the hollow adapter piece 404 during vaporization of the ingestible compound (e.g., due at least in part to the alignment of the adapter piece aperture 408 about the axis 122 and the cross-dimension of the adapter piece aperture 408 being substantially similar to the cross-dimension of the opening 118, etc.). Further, such configuration may allow a user to place the ingestible compound into the cavity 114 via the opening 118 and the adapter piece aperture 408.

The hollow adapter piece 404 may be fluidically engaged with the tube 402 at an adapter piece outlet 410 in any appropriate manner. In turn, vaporized molecules of the ingestible compound may therefore pass from the opening 118 of the electrically non-conductive bowl 102 through the hollow adapter piece 404 and into the tube 402 at the adapter piece outlet 410. The hollow adapter piece 404 may include various other components configured to direct the vaporized molecules to the tube 402 at the appropriate location. For example, the hollow adapter piece 404 may include a transfer conduit 412 fluidically engaged with the chamber 408 and the adapter piece outlet 410. In one embodiment, the transfer conduit 412 may direct the vaporized molecules to enter the tube 402 at an elevation lower than that at which the ingestible compound was vaporized, as depicted in FIGS. 4A-4C. In some cases, for example where the tube 402 is at least partially filled with water for cooling the vaporized molecules, this configuration may cause the vaporized molecules to enter the tube 402 at the adapter piece outlet 410 below a standing water line. In this regard, water held within tube 402 may also at least partially fill the transfer conduit 412 such that the vaporized molecules pass through the water (e.g., for cooling) while traveling into the tube 402 for subsequent ingestion by a user.

In this regard, the tube 402 may be defined by a cavity 414 that is configured to hold a volume of water or other liquid that may be used to cool the vaporized molecules of the ingestible compound. The tube 402 may therefore be closed across a maximum cross-dimension of the cavity 414 at a tube base 416. Further, the tube 402 may include a tube opening 418 configured to facilitate the ingestion of the vaporized molecules of the ingestible compound (e.g., via a mount piece, hose, or any other appropriate implement, etc.). According to one implementation, the tube 402 may extend along an axis substantially parallel with the axis 122 of the vaporizing assembly 100.

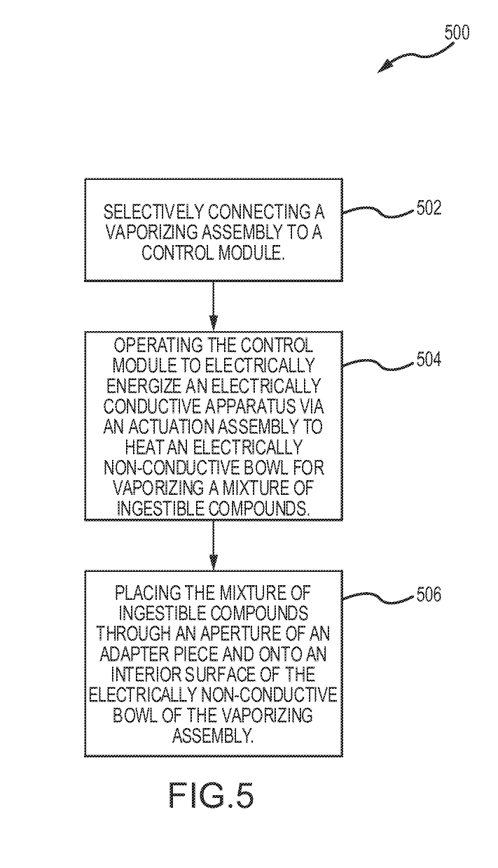

To facilitate the reader's understanding of the various functionalities of the utilities disclosed herein, reference is now made to the flow diagram in FIG. 5, which illustrates method 500 for use in vaporizing an ingestible compound. While specific steps (and orders of steps) of method 500 have been illustrated and will be discussed, other methods (including more, fewer, or different steps than those illustrated) consistent with the teachings presented herein are also envisioned and encompassed with the present disclosure.

In this regard, with reference to FIG. 5, method 500 relates generally to vaporizing an ingestible compound. The method 500 may be initiated by selectively connecting 502 a vaporizing assembly (e.g., vaporizing assembly 100) to a control module (e.g., control module 300). In this regard, the vaporizing assembly may be, for example, electrically interconnected with a power source for passing current through the vaporizing assembly to facilitate the vaporization of an ingestible compound. The method 500 may continue by operating 504 the control module to electrically energize an electrically conductive apparatus (e.g., electrically conductive apparatus 124) via an actuation assembly (e.g., actuation assembly 126) to heat an electrically non-conductive bowl (e.g., electrically non-conductive bowl 102) for vaporizing the mixture of ingestible compounds. In this regard, the mixture of ingestible compounds may be vaporized such that vaporized molecules of the ingestible compounds may pass through an adapter piece (e.g., adapter piece 404, etc.) and into a tube (e.g., tube 404) fluidically engaged with the adapter piece at an adapter piece outlet (e.g., adapter piece outlet 410). Further, the electrically conductive apparatus may be disposed over an exterior surface (e.g., exterior surface 112, etc.) of a wall (e.g., wall 104, etc.) of the electrically non-conductive bowl.

The method 500 may continue by placing 506 the mixture of ingestible compounds through an aperture (e.g., adapter piece aperture 408) of the adapter piece and onto an interior surface (e.g., interior surface 110) of the electrically non-conductive bowl of the vaporizing assembly. In this regard, the mixture of ingestible compounds may be placed within the electrically non-conductive bowl, which includes the wall having opposite first and second ends (e.g., first and second ends 106, 108) and opposite interior and exterior surfaces (e.g., interior and exterior surfaces 110, 112), an opening adjacent the first end of the wall (e.g., opening 118), a base portion adjacent the second end of the wall (e.g., base portion 116), a cavity defined by the base portion and interior surface of the wall (e.g., cavity 114), and an axis extending through the opening, the cavity and the base portion (e.g., axis 122). Further, the ingestible compounds may be placed within the electrically non-conductive bowl relative to a base portion that includes a base surface (e.g., base surface 120) that tapers from the second end of the wall towards the first end of the wall to distribute the ingestible compound received through the opening onto the base surface towards the interior surface of the wall. In this regard, the base surface may be closed across a maximum cross-dimension of the cavity.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. For instance, in some embodiments, the disclosed utilities may be configured for vaporizing a non-ingestible compound. In yet other embodiments, for example, the tube 402 may be integrally formed with the vaporizing assembly 100 to form a substantially portable apparatus for vaporizing an ingestible compound, according to the embodiments disclosed herein (e.g., such as a pipe, an electronic cigarette, or any other analogously appropriate configuration, etc.). The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

1. A disposable for vaporizing an ingestible compound, comprising:
an electrically non-conductive bowl that includes a wall having opposite first and second ends and opposite interior and exterior surfaces, an opening adjacent the first end of the wall, a base portion adjacent the second end of the wall, a cavity defined by the base portion and the interior surface of the wall, and an axis extending through the opening, the cavity and the base portion, wherein the base portion includes a base surface that tapers from the second end of the wall towards the first end of the wall to distribute an ingestible compound received through the opening onto the base surface towards the interior surface of the wall, and wherein the base surface is closed across a maximum cross-dimension of the cavity;
an electrically conductive apparatus disposed over the exterior surface of the wall of the electrically non-conductive bowl; and
an actuation assembly electrically interconnected to the electrically conductive apparatus that electrically energizes the electrically conductive apparatus to heat the electrically non-conductive bowl for vaporizing the ingestible compound.

2. The disposable of claim 1, wherein the electrically conductive apparatus is in contact with the exterior surface of the wall.

3. The disposable of claim 2, wherein the electrically conductive apparatus is wrapped over the exterior surface of the wall about the axis.

4. The disposable of claim 3, wherein the electrically conductive apparatus is a flat wire that is spiral wound over the exterior surface of the wall about the axis.

5. (canceled)

6. The disposable of claim 1, wherein the actuation assembly includes:
a first electrically conductive path electrically connected to a first end of the electrically conductive apparatus;
a second electrically conductive path electrically connected to a second end of the electrically conductive apparatus; and
an electrically conductive base member having a first cavity that receives a portion of the electrically non-conductive bowl, wherein the electrically conductive base member defines a portion of the first conductive path.

7. (canceled)

8. The disposable of claim 6, wherein the actuation assembly further includes:
at least one electrically non-conductive fastener receivable through an aperture in the portion of the bowl and an aligned bore in the base member for securing the electrically non-conductive bowl to the base member such that the electrically conductive apparatus is electrically interconnected with the first conductive path of the actuation assembly.

9. The disposable of claim 8, wherein the bore of the base member is defined by an electrically conductive inner surface that defines a portion of the first conductive path, and wherein the electrically conductive inner surface is defined by an electrically conductive insert that is received within the bore.

10. (canceled)

11. The disposable of claim 6, wherein the actuation assembly further includes:
an electrically conductive spacer member disposed between the portion of the bowl and the base member, wherein the electrically conductive spacer member defines a portion of the first conductive path, and wherein the electrically conductive spacer member is in electrical contact with the first end of the electrically conductive apparatus; and
a first conductive post that is received in a second cavity of the base member, wherein the first conductive post defines a portion of the first conductive path, the axis of the bowl extending through the first and second cavities of the base member.

12.-27. (canceled)

28. A system, comprising:
the disposable of claim 1; and
a control module electrically connected to the actuation assembly, wherein manipulation of the control module transmits an electric current through the actuation assembly to heat the electrically conductive apparatus and the bowl to vaporize the ingestible compound.

29. (canceled)

30. A system for vaporizing an ingestible compound, the system comprising:
a tube;
a hollow adaptor piece fluidically engaged with the tube at an adapter piece outlet;
a vaporizing assembly disposed within a chamber of the adapter piece and configured to receive an ingestible compound through an adapter piece aperture, the vaporizing assembly comprising:
an electrically non-conductive bowl that includes a wall having opposite first and second ends opposite interior and exterior surfaces, an opening adjacent the first end of the wall, a base portion adjacent the second end of the wall, a cavity defined by the base portion and the interior surface of the wall, and an axis extending through the opening, the cavity and the base portion, wherein the base portion is closed across a maximum cross-dimension of the cavity; and
an electrically conductive apparatus disposed over the exterior surface of the wall of the electrically non-conductive bowl and configured for vaporization of the ingestible compound; and
a control module configured to transmit electrical current to the electrically conductive apparatus;
wherein the transmission of electrical current to the electrically conductive apparatus by the control module heats the electrically non-conductive bowl to vaporize the ingestible compound at the vaporization assembly such that vaporized molecules of the received ingestible compound pass through the adapter piece and into the tube at the adaptor piece outlet.

31. The system of claim 30, wherein the control module includes a depressible actuation button, and wherein the control module is configured such that depression of the depressible actuation button activates the electrically conductive apparatus.

32.-35. (canceled)

36. The system of claim 30, wherein the vaporizing assembly further includes:
an actuation assembly electrically interconnected to the electrically conductive apparatus and the control module that electrically energizes the electrically conductive apparatus to heat the electrically non-conductive bowl for vaporizing the ingestible compound, wherein the actuation assembly includes:
a first electrically conductive path electrically connected to a first end of the electrically conductive a apparatus;
a second electrically conductive path electrically connected to a second end of the electrically conductive apparatus; and
an electrically conductive base member having a first cavity that receives a portion of the electrically non-conductive bowl, wherein the electrically conductive base member defines a portion of the first conductive path.

37.-44. (canceled)

45. The system of claim 36, wherein a longitudinal axis of the at least one fastener is perpendicular to the axis of the bowl.

46. The system of claim 36, wherein the actuation assembly further includes:
an insulating member that electrically isolates the first and second conductive paths, the insulating member being an insulating sleeve disposed about the electrically conductive apparatus.

47. (canceled)

48. The system of claim 46, wherein a gap is defined between the insulating member and the electrically non-conductive bowl, the gap being defined about a substantial entirety of a circumference of the electrically non-conductive bowl.

49. (canceled)

50. The system of claim 46, wherein the actuation assembly further includes:
a conductive member secured to an outside surface of the insulating member opposite the electrically conductive apparatus, wherein the conductive member defines a portion of the second conductive path; and
an electrically conductive lead electrically connected between the conductive member and the second end of the electrically conductive apparatus, wherein the electrically conductive lead defines a portion of the second conductive path, and wherein the electrically conductive lead extends through an opening in the insulating member.

51.-53. (canceled)

54. The system of claim 50, wherein the conductive member is a shaft, and wherein the actuation assembly further includes:
a second conductive post secured to the conducive member, wherein the electrically conductive post defines a portion of the second conductive path.

55.-56. (canceled)

57. The system of claim 30, wherein the base portion of the bowl is a conical projection.

58.-59. (canceled)

60. The system of claim 30, wherein the tube is a water pipe.

61. A method for vaporizing an ingestible compound, the method comprising:
placing a mixture of ingestible compounds through an aperture of an adapter piece and onto an interior surface of an electrically non-conductive bowl of a vaporizing assembly disposed within a chamber of the adapter piece, the electrically non-conductive bowl including a wall having opposite first and second ends and opposite interior and exterior surfaces, an opening adjacent the first end of the wall, a base portion adjacent the second end of the wall, a cavity defined by the base portion and the interior surface of the wall, and an axis extending through the opening, the cavity and the base portion, wherein the base portion includes a base surface that tapers from the second end of the wall towards the first end of the wall to distribute an ingestible compound received through the opening onto the base surface towards the interior surface of the wall, and wherein the base surface is closed across a maximum cross-dimension of the cavity; and
operating a control module to electrically energize an electrically conductive apparatus via an actuation assembly to heat the electrically non-conductive bowl for vaporizing the mixture of ingestible compounds such that vaporized molecules of the ingestible compounds pass through the adapter piece and into a tube fluidically engaged with the adapter piece at an adapter piece outlet, the electrically conductive apparatus disposed over the exterior surface of the wall of the electrically non-conductive bowl.

62. The method of claim 61, further comprising before the operating:
selectively connecting the vaporizing assembly to the control module.

63.-86. (canceled)

87. The method of claim 61, wherein the base portion of the bowl is a conical projection.

88. The method of claim 61, wherein the electrically non-conductive bowl comprises a material selected from the group consisting of quartz glass and borosilicate glass.

89. The method of claim 61, wherein the electrically conductive apparatus comprises a material selected from the group consisting of nichrome and iron-chromium-aluminum alloy.

90. The method of claim 61, wherein the operating occurs before the placing.
